(19) United States
(12) Patent Application Publication
(10) Pub. No.: US 2015/0352249 A1
(43) Pub. Date: Dec. 10, 2015

(54) Soft Calcium Hydroxyapatite & a Method for Preparation Thereof Used for Filling Gaps in Human Bone

(71) Applicant: Ghansham Das AGARWAL, Kolkata (IN)

(72) Inventor: Ghansham Das Agarwal, Kolkata (IN)

(21) Appl. No.: 14/760,262

(22) PCT Filed: Jan. 22, 2014
(86) PCT No.: PCT/IN2014/000045

§ 371 Date: Jul. 10, 2015

(30) Foreign Application Priority Data
Jan. 22, 2013 (IN) 77/KOL/2013

(51) Int. Cl. A61L 27/12; C01B 25/32; A61L 27/58

(52) U.S. Cl. CPC A61L 27/12; A61L 27/58; C01B 25/325; A61L 2430/02; A61L 2400/06

(57) ABSTRACT

Provided herein is a method for preparation of soft calcium Hydroxyapatite including: mixing a salt of calcium ion with a polymer to obtain a solution, addition of a salt of phosphate ion with the solution thus obtained to obtain aqueous calcium Hydroxyapatite, freeze drying to obtain soft calcium Hydroxyapatite (HA). The soft calcium Hydroxyapatite is a stable compound, which is sponge like with cellular matrix. When it is filled into a cavity of bone, it makes a wide area contact with bony cage around.

### FIELD OF INVENTION

This invention relates to a soft Calcium Hydroxyapatite & a method for preparation thereof used for filling gaps in human bone. The calcium Hydroxyapatite is a soft material.

### BACKGROUND OF INVENTION

Some times parts of bone are lost due to accidents, diseases or surgical operations. Conventionally bone chips from the iliac crest hip bone of the patient are used to fill the gap in the bone. This method involves another operation on the already compromised patient. Therefore, it is associated with risk of complications & problems.

Human bones are made of Calcium Phosphate in the form of a special molecular structure, which is called Calcium Hydroxyapatite. In this, Calcium is in the ratio of approximately 3.6 to 1 phosphate. In short it is called HA. Herein after in this specification it will be referred as HA. Over the years of use it is proven that HA can be safely used to fill the gaps in the bone. HA causes healing of bone over period of time by creeping substitution and forming complete bone. HA acts as an osteo-conductive material, which allows bone to heal and leads to bone growth.

There are several ways to obtain HA. Naturally it can be obtained from any bone by removing organic part of it. Conventionally, animal bone and bovine bone is used to make natural HA. There are several methods to synthesize it.

HA is normally available in form of rigid & fragile granules, blocks, powder or paste. However, it is associated with the following drawbacks:
1. Powders & granules are difficult to be accommodated in the cavity, which spill over on the surrounding tissue. Large granules do not make an intimate contact with the surrounding osseous (bone) tissue. Since it is a osteo-conductive material, intimate contact with surrounding bony tissue is required for the faster healing of bone.
2. Blocks do not spill over on the surrounding tissue, but at the same time do not make wide area intimate contact with bony tissue. This causes delay in healing.

Hence, it is required to develop a soft Calcium Hydroxyapatite for filling gaps in human bone, which can address the aforesaid drawbacks.

### OBJECTS OF INVENTION

An object of this invention is to propose a soft Calcium Hydroxyapatite & a method for preparation thereof used for filling gaps in human bone.

Another object of this invention is to propose a soft Calcium Hydroxyapatite used for filling gaps in human bone, which is soft.

Still another object of this invention is to propose a novel calcium hydroxyapatite which can be packed in cavity of bone to take shape of the cavity.

Still another object of this invention is to propose a novel calcium hydroxyapatite which can be molded by hand.

Still another object of this invention is to make HA, which does not spill out of the cavity of bone after filling.

Still another object of this invention is to propose a novel Calcium Hydroxyapatite & a method for preparation thereof used for filling gaps in human bone in which the calcium Hydorxyapatite makes wide area intimate contact with surrounding bone tissue.

### SUMMARY OF THE INVENTION

The method of preparation of calcium Hydroxyapatite comprises reaction of calcium salt with a water soluble polymer to obtain a solution. Said solution thus obtained is further subjected to reaction with phosphate salt to obtain aqueous HA, which is kept for some time. This is followed by washing with water and freeze drying so as to obtain soft calcium Hydroxyapatite (HA) of the present invention. It is soft, stable and sponge like with cellular matrix.

### STATEMENT OF INVENTION

According to this invention, there is provided a method for preparation of soft calcium Hydroxyapatite comprising steps of:
mixing a salt of calcium ion with a polymer to obtain a solution,
addition of a salt of phosphate ion with the solution thus obtained to obtain aqueous calcium Hydroxyapatite,
freeze drying to obtain soft calcium Hydroxyapatite (HA).

### DETAILED DESCRIPTION OF THE INVENTION

The present invention is directed to a novel Calcium Hydroxyapatite & a method for preparation thereof used for filling gaps in human bone. Method of preparation of soft calcium Hydroxyapatite comprising preparation of two solutions, in which a salt with calcium ion is mixed with a polymer, which is further mixed with another salt having phosphate ion. The salt with calcium ion is such as calcium Nitrate (CaNo₃), and the salt with phosphate ion is such as Diammonium phosphate (NH₄)₂HPO₄. The polymer is for example Poly vinyl alcohol without restricting scope of the invention to the same. Thus, the present invention is not restricted to the examples given hereinabove, other salt of calcium ion and phosphate ion, and other polymer readily apparent to a person skilled in the art are understood to be within the scope of the invention. The amount of calcium salt, polymer and phosphate salt is 40-60%, 10-30% and 20-40% respectively. The reaction between calcium salt and Polymer is as follows:

Calcium salt+Polymer−A solution of polymer with Calcium salt.

The solution thus obtained is further subjected to reaction with phosphate salt to obtain aqueous HA, which is as follows:

Calcium salt+Phosphate salt->Ammonium Nitrate+Aqueous HA.

Ammonium Nitrate is slowly washed out because it is soluble.

The HA thus obtained is kept for 1-15 days in still condition. Thereafter, top fluid is decanted followed by addition of fresh water, which is continued for 3-10 days. Thereafter, it is frozen at −10° C. to −30° C. followed by freeze drying at −10 to −25° C. to obtain soft calcium Hydroxyapatite (HA).

The calcium Hydroxyapatite is stable compound, which remains stable at normal room temperature.

Thus, said freeze drying gives rise a sponge like cake with cellular matrix.

When the resultant dried cake is immersed in water, it becomes soft like sponge by absorbing water. It can be pressed to take any form. When filled into a cavity of bone, it does not spill out & makes a wide area contact with bony cage around.

### Working Example

Calcium Nitrate (CaNO₃) in an amount of 50-150 gm was mixed with 10-40 gm of Poly vinyl alcohol to obtain a solution. The solution was further added with 20-60 gm of Diammonium Phosphate (NH₄)₂HPO₄.

This resulted in aqueous HA together with Ammonium Nitrate, which was slowly washed out. Thereafter, HA was kept for 12 days in still condition. This was followed by decantation of top fluid followed by addition of fresh water for 9 days. Then it was frozen at −10° C. followed by freeze drying at −10° to obtain soft calcium Hydroxyapatite (HA).

### Advantageous Features

Soft material to take shape of the cavity in bone.

Does not spill out of the cavity after filling.

Wider area intimate contact with surrounding bone tissue:

Reliable for use.

It is to be noted that the present invention is susceptible to modifications, adaptations and changes by those skilled in the art. Such variant embodiments employing the concepts and features of this invention are intended to be within the scope of the present invention, which is further set forth under the following claims:

1. A method for preparation of soft calcium Hydroxyapatite comprising steps of
mixing a salt of calcium ion with a polymer to obtain a solution,
adding a salt of phosphate ion to the solution to obtain aqueous calcium Hydroxyapatite,
freeze drying the aqueous calcium Hydroxyapatite to obtain soft calcium Hydroxyapatite (HA).

2. The method as claimed in claim 1, wherein the salt of a calcium ion is calcium Nitrate (CaNO₃) having an amount of 40-60%.

3. The method as claimed in claim 1, wherein the salt of a phosphate ion is Diammonium phosphate (NH₄)₂HPO₄ having an amount of 20-40%.

4. The method as claimed in claim 1, wherein the polymer is Poly vinyl alcohol having an amount of 10-30%.

5. The method as claimed in claim 1, wherein the aqueous calcium Hydroxyapatite is kept for 1-15 days in still condition followed by decantation.

6. The method as claimed in claim 5, wherein the decantation is followed by addition of fresh water and storage for 3-10 days.

7. The method as claimed in claim 1, wherein freeze drying is carried out at −10 to −25° C. after freezing at −10 to −30° C. to obtain soft calcium Hydroxyapatite (HA).

8. The method as claimed in claim 1, wherein the calcium Hydroxyapatite, when immersed in water, becomes soft by absorbing water.

9. Soft calcium Hydroxyapatite produced by the method according to claim 1.
